(19) United States
(12) Patent Application Publication
(10) Pub. No.: US 2016/0045686 A1
(43) Pub. Date: Feb. 18, 2016

(54) SPACER CHAMBER

(71) Applicant: Diego Jaroslavsky, Ciudad Autonoma de Buenos Aires (AR)

(72) Inventor: Diego Jaroslavsky, Ciudad Autonoma de Buenos Aires (AR)

(21) Appl. No.: 14/458,297

(22) Filed: Aug. 13, 2014

(51) Int. Cl. A61M 15/00

(52) U.S. Cl. CPC A61M 15/0088; A61M 15/009; A61M 15/0021; A61M 15/002; A61M 2207/00

(57) ABSTRACT

The present invention is directed to a spacer chamber having a chamber having a truncated cone shaped or two truncated cones joined by their bases. The truncated cones are formed by a series of popoids segments that allow the chamber to keep its shape, both on an extended and a collapsed position, in a spontaneous form, except in the case of an internal pressure is applied which tends to expand it. As is the case of the device used in mechanical ventilation.

### FIELD OF THE INVENTION

The present invention refers to a spacer chamber having the form of one or two truncated cones joined by their bases. The truncated cones include zigzag type pleated popoides that decrease in size, allowing the spacer chamber to be easily collapsed or extended, for the administration of the therapeutic agent in aerosol form. The spacer chamber can be used both personally and on a mechanical breathing machine.

### BACKGROUND INFORMATION

A series of spacer chambers having different shapes and mechanisms are commercially known to facilitate the pharmaceutical drug administration by respiratory inhalation. The majority of them being variations of the ones used in children or adults.

The best known are the Aerochamber, which has two valves to prevent the loss of aerosol during expiration, the Volumatic, which is for adults and children, and the Babyhaler which also has valves.

The problem of some of these spacer chambers is they are made of plastic, a material that generates a lot of static electricity and attracts the aerosol particles; for this reason, in Sweden a stainless steel spacer chamber was created, which is a material that is not charged with static electricity and prevents the adhesion of particles. In some plastic chambers an additive was added that makes them antistatic.

Many types of spacer chambers have been developed with different volumes and sizes. Many of them being of great size and difficult to assemble, especially at the time when the patient needs to use them.

The chambers having large volumes are also of large size, making it difficult for their transportation in the case of being personally used, which has the risk of being un-operative by cracks and tears on the plastic due to hits or pressures.

In addition, collapsible spacer chambers have been developed, with different types of structures, being the bellows type (accordion) or with rigid rings, the most common, but that at the time of use, may be complicated because they are difficult to properly extend, especially at the time the patient needs the medication.

Furthermore, spacer chambers are being developed to be used by ambulatory patients in conjunction with breathing circuits for patients on mechanical ventilation, rigid, as well as collapsible. The collapsible systems are generally formed by cylindrical tubes made of a soft material that requires them to be held in order to keep them in a proper position.

The popoides system has been used in connectors for tubular medical devices, and, more specifically, for respiratory connectors but never on spacer chambers or spacer chambers having a truncated cone shape.

### SUMMARY OF THE INVENTION

The three general factors that alter the arrival of a drug spray to the lung are: the particle; the host, and the inhaler. Among these are the physical characteristics of the particle, the way the particle floats in the air, the particle density, its charge, the hygroscopic, the shape, and the speed. The factors which depend on the host are: inspiratory volume of the individual and the inspiratory time. The inhaler factors are a mixture of the two previous and to reduce errors, the spacer chambers have been created, whose main objectives are to promote the deceleration of the particles allowing the useful particles to be separated from the propellant and to settle better; another advantage of the creation of the spacer chambers is to provide more time for the evaporation of the propellant, which decreases the Freon cooling effect; improve the dosage especially when using corticoids (produces oral candidiasis and dysphonia).

The objective of the present invention is to provide a spacer chamber that is adequate for the pharmaceutical drug dispersion so that it conveniently arrives to the patient; to be easy to use in ambulatory patients or by professionals in mechanical ventilation. In addition to provide a semi-rigid chamber in its extended form, keeping its shape by itself.

It is also important that the spacer chamber be able to change both the size, as well as the volume occupying very little space when it is transported. Another feature is that it is easy to disassemble and easy to clean.

In summary, the present invention provides solutions for:

The chamber to be used directly by both an outpatient, as well as a patient connected to a respirator with the same medication.

The chamber is kept rigid in a collapsed or an extended position so that it is stable without the need of safety interlocks.

The drug particles are dispersed without adhering to the wall.

Provide a proper drug dispersion into the chamber so that it conveniently arrives to the patient.

Be easy to disassemble and clean.

May be able to change both the size, as well as the volume, to provide chambers of any volume while maintaining the ability to collapse to a small percentage of its size.

Be economic in marketing, sold in folded form, and by its smaller size, decrease the packaging and freight cost.

It is possible to sterilize in a family-type microwave.

### BRIEF DESCRIPTION OF THE INVENTION

The present invention includes a truncated cone shaped duct or two truncated cones joined by their bases. The truncated cones are formed by a series of POPOIDES segments that allow the chamber to keep its shape, both on an extended and a collapsed position, in a spontaneous form, except in the case of an internal pressure is applied, which tends to expand it. As is the case of the device used in mechanical ventilation.

The spacer chamber of the present invention includes a duct having a truncated cone shape or two truncated cones joined by their bases, made of a flexible material, with a plurality of popoids folds, type zigzag, which circumferentially are formed in the wall of the space chamber in a decreasing size pattern from the base. When the chamber is collapsed each segment of it fits entirely inside the adjacent section i.e. is closed and occupying a minimum space.

The duct to the chamber in the extended, as in the collapsed position, has the characteristic that remains more rigid than any other duct of the same material or heavier material.

The spacer chamber has in one of the ends close to the cone base, is connected through a device that contains a one-way valve, with an anatomical mask used by the patient. At the other end of the chamber and through the other device connects with the spray containing the drug. The space chamber can also be used in professional equipment for breathing mechanics with different couplings.

The ends of the chamber may be assembled with fasteners in different ways: screw up to each other, or can be connected by pressure or glue or by other methods such as ultrasound or thermo fusion.

Also within the present invention, the system allows other economical and simple versions. One that has built-in one piece with the chamber and of the same material, a direct connector for the MDI. Or another version which is when the spacer chamber has the ends of one piece on which is only added to the one-way valve, with couplings and the assembled ends by means of threads, joined by pressure, or joined by ultrasound or thermo fusion.

The space chamber has the form of a bellows truncated cone with pleated popoides descending from the base to the tip. It may also be in the form of two truncated cones joined by their bases. The zigzag folds are folds formed with the popoides system, i.e. that are kept rigid both in the extended, as well as the collapsed positions, allowing mechanical ventilation in people connected to respirators as the chamber is pleated allows the gaseous fluid to pass directly without modify, directly and without changes, and in its extended form to administer the medication.

### BRIEF DECRIPTION OF THE DRAWINGS

FIG. 1 is a perspective view of the spacer chamber according to an embodiment of the present invention showing the devices 2 and 3 embedded;

FIG. 2 is a side view of the spacer chamber with the embedded devices of FIG. 1;

FIG. 3 is a front view of the spacer chamber of FIG. 1;

FIG. 4 is a longitudinal cut view of the expanded chamber without couplings;

FIG. 5 is a longitudinal cut view of the collapsed or folded chamber;

FIG. 6 is a perspective view of a double spacer chamber according to an embodiment of the present invention;

FIG. 7 is a side view of the double spacer chamber of FIG. 6;

FIG. 8 is a side view of the double spacer chamber collapsed or folded;

FIG. 9 is a perspective front view of the double spacer chamber showing incorporated a direct connector for MDI;

FIG. 10 is a perspective front view of the double spacer chamber having built-in threaded ends;

FIG. 11 shows a side view of the spacer chamber according to one embodiment of the present invention showing a spacer chamber for outpatient treatment showing the chamber in an extended position;

FIG. 12 shows a left side view of the spacer chamber of FIG. 11;

FIG. 13 shows a cross sectional view of the spacer chamber taken alone line A-A of FIG. 12 showing the chamber in a collapsed position;

FIG. 14 shows a side view of the spacer chamber according to one embodiment of the present invention showing a spacer chamber for use with mechanical ventilation;

FIG. 15 shows a left side view of the spacer chamber of FIG. 14;

FIG. 16 shows a top view of the spacer chamber of FIG. 14; and

FIG. 17 shows a cross sectional view of the spacer chamber taken alone line A-A of FIG. 16.

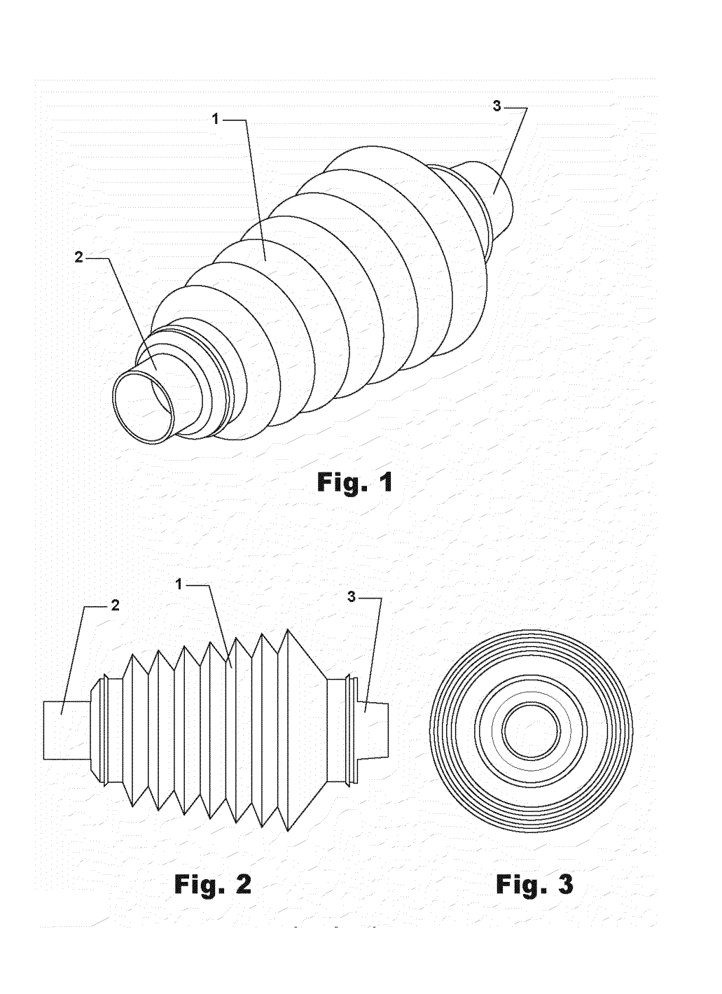

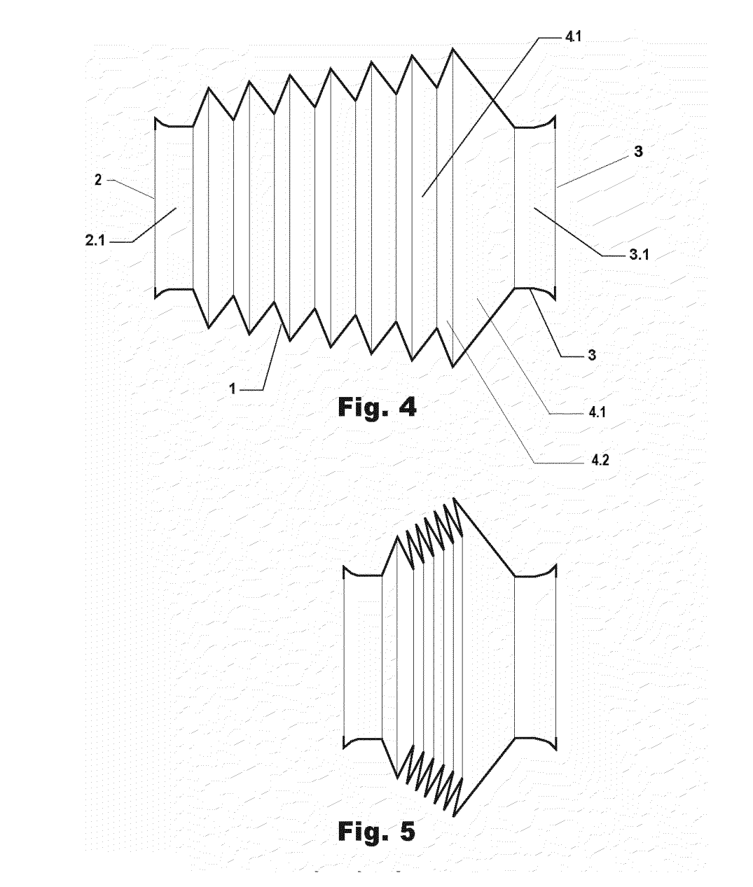

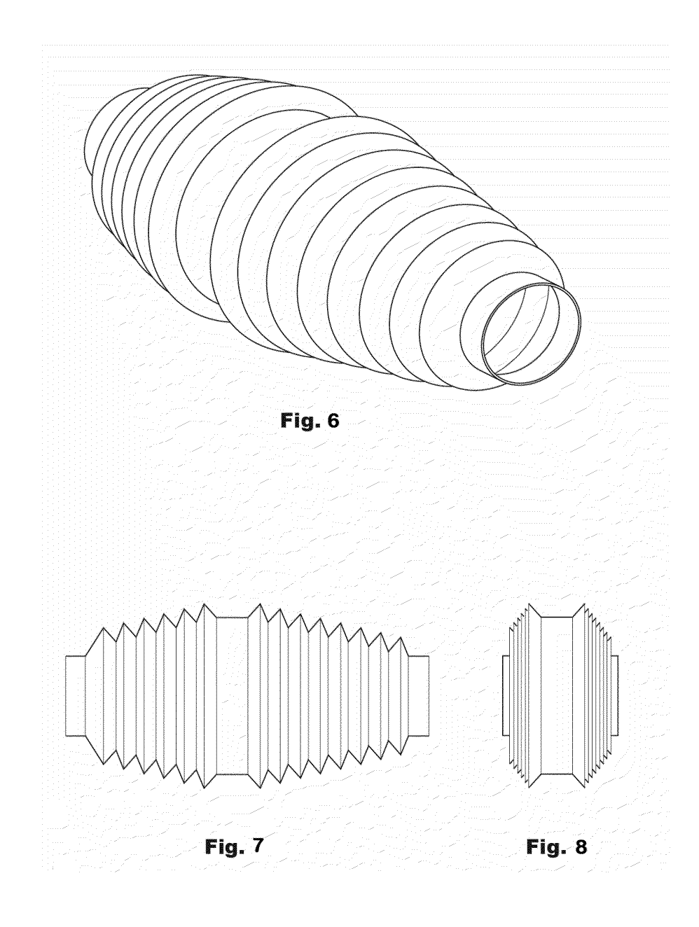

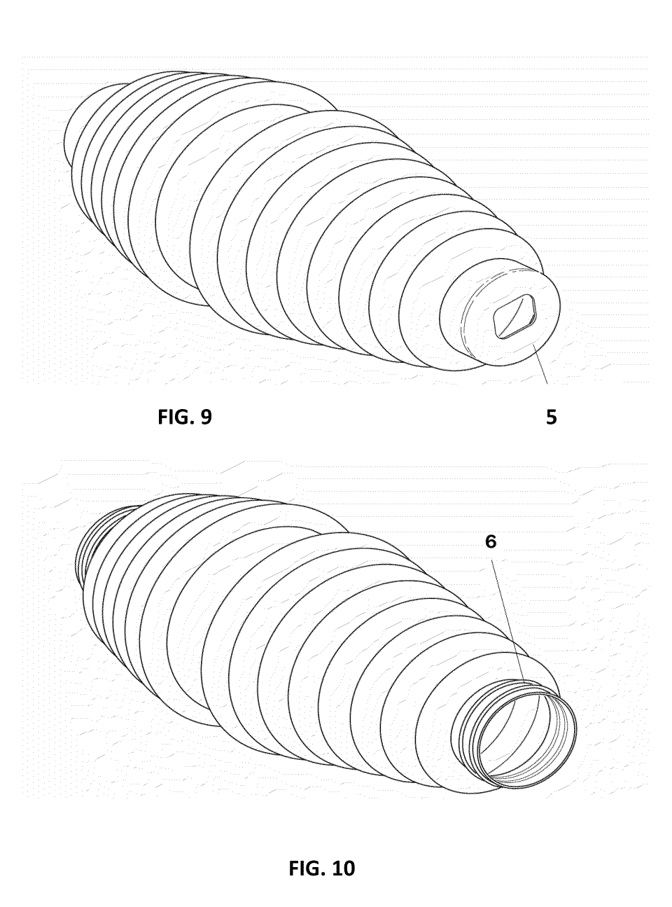

### DESCRIPTION OF COMPONENT PARTS

1. Chamber with bellows.
2. main mouth for the drug access.
2.1 main mouth diameter.
3. secondary or suction mouth.
3.1 secondary mouth diameter.
4. folds inside the chamber, unevenly or progressive enlargement.
4.1 external fold.
4.2. inner fold.
5—direct connector for the MDI.
6. chamber with screw terminal.
10. chamber for outpatient treatment spacer chamber
12. Mask
14. Exhalation Valve
16. threaded support
17. fasteners
18. thread
19. aspiration Valve
20. chamber for mechanical ventilation spacer chamber
22. female connector
24. male connector
26. aerosol input device aerosol device

### DETAILED DESCRIPTION OF THE INVENTION

The spacer chamber of the present invention comprises a duct with a truncated cone shape or two truncated cones joined by bases, made of flexible material as i.e. polypropylene or polyethylene, with a plurality of zigzag popoid sections, that when it is collapsed each popoid segment is against the next section and fits entirely inside the adjacent section; thus, it remains rigid in both the extended or the collapsed position.

In a cross section of the chamber, it can be seen that the truncated cone is formed by a succession of folds in the form of declining zigzag from the base of the cone to the vertex.

The chamber material has anti-static properties, thus, the particles in suspension do not stick to it.

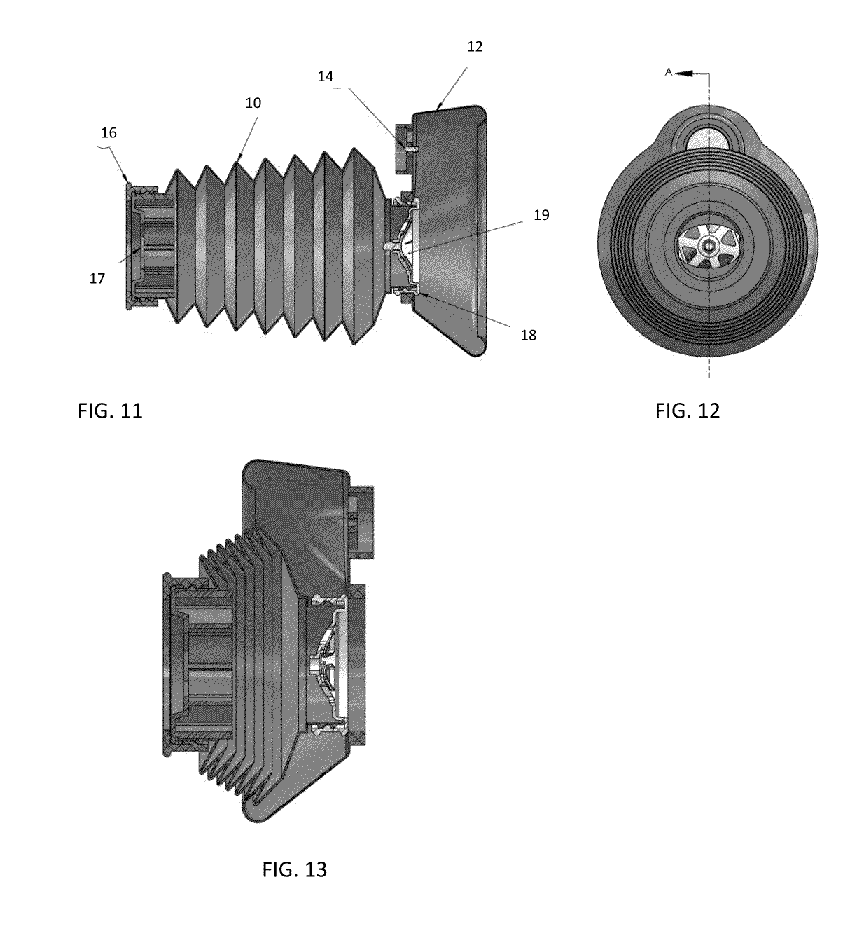

FIGS. 11-13 show the spacer chamber according to one embodiment of the present invention showing a spacer chamber for outpatient treatment. The spacer chamber includes a chamber 10 having the form of bellows is showed in an extendable position; a mask 12 for the patience including a exhalation valve 14; threaded support 16 for the aerosol including fasteners 17; a thread 18 and a aspiration valve 19.

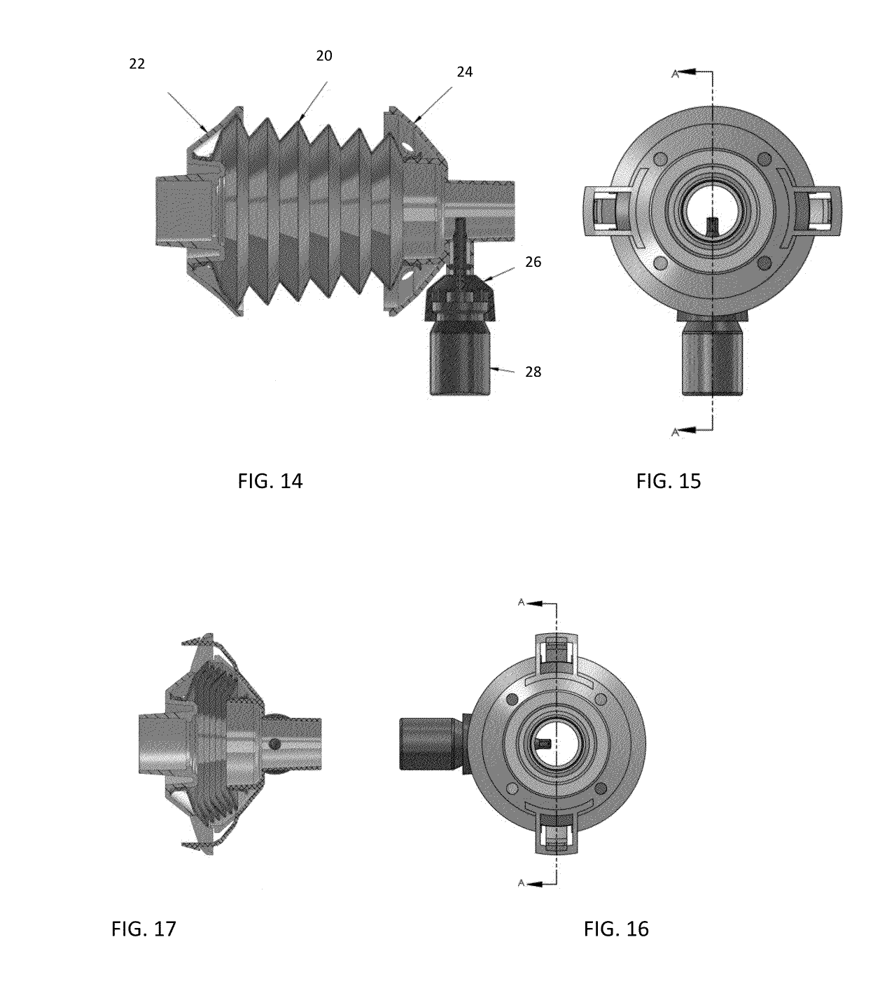

FIGS. 14-17 shows a side view of the spacer chamber according to one embodiment of the present invention showing a spacer chamber for use with mechanical ventilation. The spacer chamber includes the chamber 20 having bellows having the shape of a truncate cone; female connector 22 with output to the patient including clamps to prevent the chamber to extend because of the pressure of the ventilator; male connector 24 for air input having clamps for the aerosol; aerosol input device 26; and aerosol device 28.

What is claimed is:

1. A spacer chamber for administering a therapeutic agent spray comprising:
a chamber having a base and a tip, the chamber is made of a flexible material, the chamber has a truncated cone shape or two truncated cones joined at their bases, a plurality of popoides folds though the chamber, the popoides decreasing in size from the base to the tip.

2. The spacer chamber of claim 1, wherein the flexible material has antistatic properties.

3. The spacer chamber of claim 1, further including a device located at the tip of the chamber, the device having a first side adapted to be connected to a mask or a nozzle and a second side adapted to be connected to a connector to attach a container with the medication.

4. The spacer chamber of claim 3, wherein the ends of the chamber are joined to fittings, threads or pressure or stuck by ultrasound or hot melt.

5. The spacer chamber of claim 1, further including a built-in piece on the tip of the chamber, the built in piece is adapted to be directly connected to a MDI device.

6. The spacer chamber of claim 4, wherein the fittings include locks.
